(19) United States
(12) Patent Application Publication
(10) Pub. No.: US 2008/0039613 A1
(43) Pub. Date: Feb. 14, 2008

(54) Process for the Production of Azidoalkylamines

(76) Inventor: Jan Haller, Leverkusen (DE)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/547,722

(22) PCT Filed: Apr. 7, 2005
(86) PCT No.: PCT/EP05/03655

§ 371 Date: Aug. 29, 2007

(30) Foreign Application Priority Data
Apr. 7, 2004 (DE) 10 2004 017 607.8
Sep. 20, 2004 (DE) 10 20047 045 870.

(51) Int. Cl. C07C 247/04

(52) U.S. Cl. 534/555

(57) ABSTRACT

The invention relates to a method for producing azidoalkyl amines. According to said method, in a first step a bifunctional alkane comprising two different nucleofugic groups is reacted with an azide to form a monofunctional azidoalkane, during which process one nucleofugic group is cleaved and in a second step, the monofunctional azidoalkane is reacted with a primary or secondary amine to form azidoalkyl amine, during which process the second nucleofugic group is cleaved.

The invention relates to a process for the production of azidoalkylamines.

Azidoalkylamines play an important role as intermediates in organic synthesis.

The synthesis of alkyl azides by nucleophilic substitution is known. In this synthesis, the appropriate starting compound reacts with an azide.

In J. Org. Chem. 1957, 22, 238-240, the synthesis of alkyl azides starting from alkyl bromides and iodides in alcohols or 2-alkoxyethanols with the addition of water is described. The alkyl azides do not carry any additional functional groups.

In Tetrahedron Lett. 1991, 32, 183-186, the production of diazidoalkanes from the corresponding dichloro or dibromo derivatives in dimethylformamide is described.

In J. Org. Chem. 1957, 22, 995-996, the synthesis of 2-chloroethyl azide starting from 2-chloroethyl-p-toluenesulfonate in a mixture of methanol and water is described. The reaction mixture is kept under reflux for 24 hours and the product is then extracted and distilled.

In J. Org. Chem. 1993, 58, 3736-3741, the synthesis of azidoalkylamines and N-(azidoalkyl)diaminoalkanes is described, wherein N-(haloalkyl)amines or -diaminoalkanes are used as intermediates. These have the disadvantage that their synthesis is complicated and that they can cyclise by intramolecular substitution. In the synthesis of N-(4-azidobutyl)-1,3-propanediamine in particular, the yield in the last step is only 30% because the ring-closing nucleophilic substitution to form N-(3-aminopropyl)pyrrolidine comes to the fore. In addition, the synthesis of azidobutylamines by aminolysis of 1-azido-4-iodobutane is described. This is in turn produced in two steps by the reaction of 1-bromo-4-chlorobutane with sodium azide to form 1-azido-4-chlorobutane and the activation of the nucleofugic group chloride by exchanging it with iodide.

The synthesis of azidoalkylamines is particularly problematic when other positions on the amino group can be substituted. Either a poor yield or costly protective group technology has to be accepted.

JP 2002-332274 describes such a synthesis, in which the amino groups are protected. The synthesis becomes very costly as a result of this and the yield over all the steps is less than 40%.

The object of the invention is to overcome the disadvantages of the prior art and, in particular, to create a process for the production of azidoalkylamines which provides good yields with relatively low costs.

The object is achieved by a process with the characteristics of the main claim. In the first process step thereof, a bifunctional alkane with two different nucleofugic groups is reacted with an azide, with the elimination of one nucleofugic group, to form a monofunctional azidoalkane. In the second step, the monofunctional azidoalkane is reacted with a primary or secondary amine with the elimination of the second nucleofugic group to form the azidoalkylamine.

The term ‘bifunctional alkane’ here means a compound that contains two groups with different nucleofugicity, in which each of the two nucleofugic groups is bound to a primary, secondary or tertiary alkyl group and the two alkyl groups are bonded to one another directly or via one or more connecting members Z. Connecting members Z can be e.g.: saturated or unsaturated hydrocarbon chains, aryl groups, saturated or unsaturated carbocycles, which can additionally contain bridging members such as e.g. —O—, —S—, —C(═O)—, —CO₂—, —SO₂— and non-reactive substituents, such as ester, amide or ether groups.

Surprisingly, it has been found that the monofunctional azidoalkane obtained in the first step (e.g. an azidochloroalkane) reacts with a primary or secondary amine without the need for a previous exchange reaction (e.g. the only slightly nucleofugic group chlorine is exchanged with a more reactive group such as iodine). It is also surprising that, in the reaction of the monofunctional azidoalkane with an amine, not the double azidoalkylated product but almost exclusively the desired single azidoalkylated product is formed.

The course of the reaction can be shown diagrammatically as follows (bifunctional alkane here contains as connecting members Z only unbranched, saturated hydrocarbon chains, second step alternatively with a primary or secondary amine):

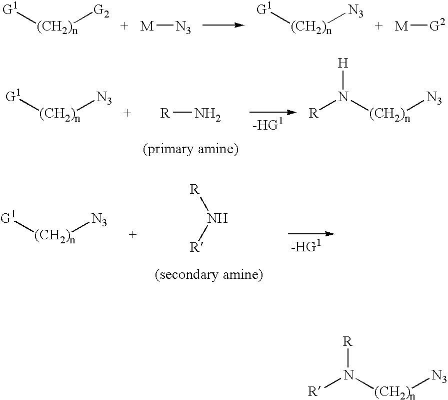

with:

G¹=nucleofugic group 1

G²=nucleofugic group 2

n=natural number from 1 to 20, preferably from 2 to 10, particularly preferably from 3 to 6.

M=metal, preferably monovalent metal such as Li, Na, K, Rb, Cs (in the case of a polyvalent metal, the formula MN₃ should be adapted as appropriate, e.g. M(N₃)₂ in the case of a divalent metal such as Ba, Zn, or M(N₃)₃ in the case of a trivalent metal such as Al); ammonium, optionally also with organic substituents, up to four H atoms of the ammonium ion possibly also being substituted by optionally different organic radicals, e.g. methyl, ethyl, propyl, butyl, benzyl), e.g. (NMe₃H)⁺, (NEt₃H)⁺, (NBu₄)⁺ or (NMe₃CH₂Ph)⁺ or with M=substituted metal or semimetal, e.g. MN₃=SiMe₃N₃, SnBu₃N₃, AlEt₂N₃.

R, R′=alkyl with 1 to 20, preferably 2 to 10, particularly preferably 3 to 6 C atoms or functionalised alkyl with 1 to 20, preferably 2 to 10, particularly preferably 3 to 6 C atoms, wherein R and R′ can also be bonded to form a ring, with for example the following functional groups (non-limiting list):

amine,

—OR¹ with {R¹=H, alkyl, alkenyl, alkynyl, aryl, heterocycle, —C(═O)R², —C(═S)R² with [R²=alkyl, alkenyl, alkynyl, aryl, heterocycle, alkoxy, aryloxy, heterocyclyloxy, —NR³R⁴ with (R³ and R⁴ independently of one another =H, alkyl, alkenyl, alkynyl, aryl, heterocycle, wherein R³ and R⁴ can also be linked to form a ring), —SR⁵ with (R⁵=alkyl, alkenyl, alkynyl, aryl, heterocycle)]}, —NR⁶R⁷ with R⁶ and R⁷ independently of one another =R¹ as defined above and, in addition, —SO₂R⁵, wherein R⁶ and R⁷ can also be linked to form a ring; —N═CR⁸R⁹ with R⁸ and R⁹ independently of one another =R² as defined above, wherein R⁸ and R⁹ can also be linked to form a ring,

—SR¹⁰ with R¹⁰=R¹ as defined above but without H; —C (═O)R¹¹, —C(═O)C(═O)R¹¹, —C(═NOR³)R¹¹, —SOR¹¹, —SO₂R¹¹, —P(═O)R¹¹R¹² with R¹¹ and R¹²=R² as defined above and additionally —OH;

—BR¹³R¹⁴ with R¹³ and R¹⁴ independently of one another =R⁵, OR³, NR³R⁴ as defined above;

—SiR¹⁵R¹⁶R¹⁷ with R¹⁵, R¹⁶ and R¹⁷ independently of one another =R⁵, OR³;

—SnR¹⁸₃ with R¹⁸=alkyl;

—F; —CN; —N₃; —NO₂;

alkenyl, alkynyl, aryl or heterocycle.

Alternatively, a compound belonging to the following general formula can also be used as the bifunctional alkane:

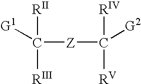

with R^{II}, R^{III}, R^{IV} and R^{V} independently of one another H, alkyl, aryl, also linked together to form a ring;

Z=saturated or unsaturated hydrocarbon chains with 1 to 20 C atoms, preferably 1 to 4 C atoms, aryl groups, saturated or unsaturated carbocycles, which additionally [can contain]

bridging members such as —O—, —S—, —C(═O)—, —CO₂—, —SO₂— and non-reactive substituents such as ester, amide or ether groups. Z can also be omitted, in which case the two C atoms in the above formula are bonded directly to one another.

Nucleofugic groups (substituents) can be e.g.: chloride, bromide, iodide or sulfonates (such as e.g. methanesulfonate, trifluoromethanesulfonate, p-toluenesulfonate). The nucleofugic group with the higher reactivity is substituted in the first reaction step (process step) by the azide group, while the nucleofugic group with the lower reactivity is only substituted in the second reaction step (process step) by the primary or secondary amine. Examples of bifunctional alkanes are 1-bromo-2-chloroethane, 1-bromo-3-chloropropane, 1-bromo-3-chloro-2-methylpropane, 1-bromo-4-chlorobutane, 1-bromo-5-chloropentane, 1-bromo-6-chlorohexane, 2-bromo-1-chloropropane, 1-bromo-3-chloro-2,2-dimethoxypropane, 1-chloro-3-iodopropane, 1-chloro-4-iodobutane, 1-chloro-5-iodopentane, 1-chloro-6-iodohexane, 2-chloroethyl p-toluenesulfonate, 2-chloroethylmethanesulfonate, 3-chloropropyl p-toluenesulfonate. An example of a bifunctional alkane with an aryl connecting member is 2-(chloromethyl)benzyl bromide.

The first step of the process is preferably carried out in a polar solvent, such as e.g. formamide, dimethylformamide, dimethylacetamide, dimethyl sulfoxide, diethylene glycol, methanol, acetone or water. Mixtures of these solvents, particularly of an organic solvent and water, can also be used. A mixture of this type preferably contains no more than 30 wt. % water. Those solvents in which both the bifunctional alkane and the azide are at least 1 wt. % soluble are particularly preferred.

The azidoalkane still containing a nucleofugic group can—but does not have to —be purified before it reacts with the primary or secondary amine to form the azidoalkylamine. In this case the intermediate compound is preferably extracted from the polar solvent of the first step with a non-polar solvent. An addition of water after the addition of the non-polar solvent is particularly preferred here.

In the second process step, the primary or secondary amine is preferably used in excess. The excess is particularly preferably 1 to 5000 mole %, for monoamines preferably 10 to 100 mole %, and for polyvalent amines, such as diamines, preferably 100 to 1000 mole %. Particularly when the primary amine is a diamine and only one amino group is to be azidoalkylated, an excess of the diamine is used or this is simultaneously used as a solvent. Water is preferably added before, during or after the reaction with the amine, or an aqueous amine is used. The quantity of water present during the reaction is preferably 5 to 90 wt. % of the total quantity of amine and water, particularly preferably 15 to 50 wt. %.

To isolate the azidoalkylamine, it can be separated from the excess amine by extraction with a non-polar solvent and distilled. The azidoalkylamine can be precipitated as a salt by adding an acid, and further purified by recrystallisation. In particular, N-(azidoalkyl)diaminoalkanes can be worked up in this way. The azidoalkylamine is preferably fractionally precipitated by first precipitating the excess unreacted amine with the necessary quantity of acid and separating it off before the azidoalkylamine is precipitated with acid.

The azidoalkylamine obtained can be purified by chromatography as a free base, as a salt of the acid released during the reaction in the second step or as a salt from the precipitation with an acid.

The azidoalkylamine obtained can also be separated and/or purified by distillation.

The invention will be explained in more detail on the basis of the following examples, without limiting it:

### EXAMPLE 1

### Production of N-(4-azidobutyl)-1,3-diaminopropane with the addition of water and fractionated precipitation as N-(4-azidobutyl)-1,3-propanediammonium bismethanesulfonate

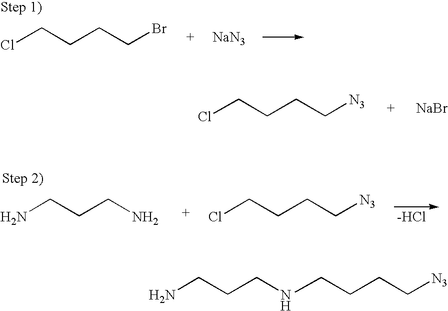

65 g of 1-bromo-4-chlorobutane were metered at room temperature into 27.1 g of sodium azide in 455 ml of dimethyl sulfoxide. After stirring overnight at room temperature, a suspension was formed to which 250 ml of hexane and 455 ml of water were added. After the phase separation, the aqueous phase was extracted with a further 125 ml of hexane. The combined organic phases were washed with 250 ml of water, and 140.5 g of 1,3-diaminopropane were added. After metering in 40 ml of water, the hexane was distilled off at 35° C. under vacuum and the mixture was stirred for a further 5 hours at 35° C. The cooled reaction mixture was extracted with 390 ml and 130 ml of toluene consecutively. 515.5 g of toluene extract were obtained. From 250 g of the toluene phase, the major part of the 1,3-diaminopropane corresponding to the analytically determined content was fractionally precipitated as 1,3-propanediammonium bismethanesulfonate by adding 16.9 g of methanesulfonic acid. The solid was filtered off and washed with 30 ml of toluene and dried. 24.2 g of a colourless solid were obtained, which contained 0.9 wt. % N-(4-azidobutyl)-1,3-propanediammonium bismethanesulfonate. From the combined filtrates, by adding 22.3 g of methanesulfonic acid, the main product was precipitated, filtered off and washed with 30 ml of toluene and twice with 30 ml of isopropanol each time. After drying the solid, 39.6 g of N-(4-azidobutyl)-1,3-propanediammonium bismethanesulfonate were obtained as a colourless solid with a purity of 98.2 wt. % (58% yield). By acidifying the filtrate to pH 3 with a further 7.7 g of methanesulfonic acid, filtering, washing the solid with toluene and isopropanol and drying, it was possible to obtain from the filtrate a further 11.5 g of product with a content of 80.6 wt. % (14% yield) N-(4-azidobutyl)-1,3-propanediammonium bismethanesulfonate.

300 MHZ-¹H-NMR (CD₃OD): δ=1.65-1.74 (m, 2H); 1.76-1.86 (m, 2H); 2.05-2.16 (m, 2H); 2.73 (s, 6H); 3.05-3.17 (m, 6H) ; 3.41 (t, 2H)

IR (KBr): v̅ (cm⁻¹)=3425, 2947, 2781, 2098, 1504, 1196, 1161, 1057, 783, 563, 536.

Melting point (DSC): 115° C.; decomposition (DSC): 217° C. (onset).

Warning: the 1-azido-4-chlorobutane isolated from the hexane extract by distilling off the solvent has an extremely high decomposition energy of 4000 J/g and is sensitive to impact!

### EXAMPLE 2

### Production of N-(4-azidobutyl)-1,3-propanediamine with the addition of water and precipitation as N-(4-azidobutyl)-1,3-propanediammonium bismethanesulfonate

65 g of 1-bromo-4-chlorobutane were metered at room temperature into 27.1 g of sodium azide in 260 ml of dimethyl sulfoxide. After stirring for two hours at room temperature, a suspension was formed to which 260 ml of hexane and 260 ml of water were added. After the phase separation, the aqueous phase was extracted with a further 130 ml of hexane. The combined organic phases were washed with 260 ml of water, and 140.4 g of 1,3-diaminopropane were added. After metering in 40 ml of water, the hexane was distilled off at 35° C. under vacuum and the mixture was stirred for a further 4 hours at 35° C. The cooled reaction mixture was extracted with 390 ml and 130 ml of toluene consecutively and the product was precipitated from the toluene phase by adding 120 g of methanesulfonic acid. The solid was washed with 130 ml of isopropanol and extracted with 730 ml of hot isopropanol. From the cooled extract, 69.4 g (56% yield) of N-(4-azidobutyl)-1,3-propanediammonium bismethanesulfonate crystallised as a colourless solid with a purity of 95%. It was possible to purify the product further by recrystallising from isopropanol/water.

### EXAMPLE 3

### Production of N-(4-azidobutyl)-1,3-propanediamine using aqueous 1,3-diaminopropane and precipitation as N-(4-azidobutyl)-1,3-propanediammonium bismethanesulfonate

5 g of 1-bromo-4-chlorobutane were metered at room temperature into 1.9 g of sodium azide in 20 ml of dimethyl sulfoxide. After stirring overnight at room temperature, a suspension was formed to which 20 ml of hexane and 20 ml of water were added. After the phase separation, the aqueous phase was extracted with a further 20 ml of hexane. The combined organic phases were washed with 20 ml of water and added to 9.7 g of 1,3-diaminopropane, which contained approx. 20 wt. % water. The mixture was stirred for 1 hour at room temperature, heated to 35° C., the hexane was distilled off under vacuum and the mixture was stirred for a further 4 hours at 35° C. The cooled, cloudy solution was extracted twice with 10 ml of toluene each time and the product was fractionally precipitated from the toluene phase by adding 5.4 g of methanesulfonic acid. The solid was washed with acetonitrile and recrystallised from 50 ml of isopropanol. 6.0 g (56% yield) of N-(4-azidobutyl)-1,3-propanediammonium bismethanesulfonate were obtained as a colourless solid with a purity of 98.4%.

### EXAMPLE 4

### Production of N-(4-azidobutyl)-1,3-propanediamine and precipitation as N-(4-azidobutyl)-1,3-propanediammonium bismethanesulfonate

20 g of 1-bromo-4-chlorobutane were metered at 35° C. into 8 g of sodium azide in 80 ml of dimethyl sulfoxide. After stirring for two hours at 35° C., a suspension was formed to which 77 ml of hexane and 77 ml of water were added after cooling to room temperature. After the phase separation, the aqueous phase was extracted with a further 40 ml of hexane. The combined organic phases were washed with 80 ml of water, and 43.2 g of 1,3-diaminopropane were added. The mixture was heated to 35° C., the hexane was distilled off under vacuum and the mixture was stirred for a further 4 hours at 35° C. During this operation, a colourless solid crystallised out. The cooled mixture was treated twice with 40 ml of toluene each time, separating off the solid consisting mainly of 1,3-propanediamine hydrochloride. The product was precipitated from the toluene phase by adding 21.5 g of methanesulfonic acid. The solid was washed with 25 ml of toluene and 50 ml of isopropanol consecutively and taken up in 450 ml of hot isopropanol. The insoluble components were filtered off. After cooling the filtrate, 19.8 g (56% yield) of N-(4-azidobutyl)-1,3-propanediammonium bismethanesulfonate were obtained therefrom as a colourless solid with a purity of 90.8% and further purified by recrystallising from isopropanol.

### EXAMPLE 5

### Production of 4-azidodibutylamine hydrochloride

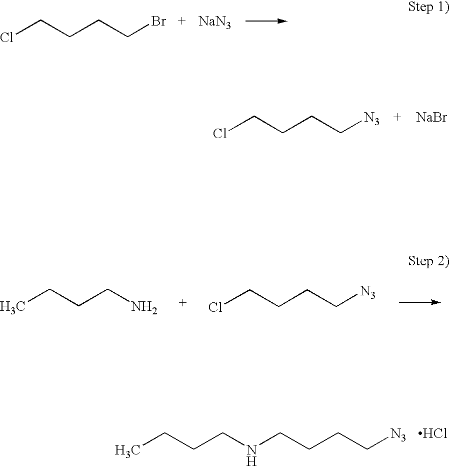

25 g of 1-bromo-4-chlorobutane were metered at 25 to 28° C. into 10 g of sodium azide in 175 ml of dimethyl sulfoxide. After stirring overnight at room temperature, 100 ml of hexane and 175 ml of water were added to the mixture, the separated aqueous phase was extracted with 50 ml of hexane and the combined organic phases were washed with 100 ml of water. 113.4 g of a solution of 1-azido-4-chlorobutane in hexane were obtained. 3.3 g of butylamine and 10 ml of ethanol were added to 17.5 g of this solution and the hexane was distilled off at 30° C. under vacuum. After stirring for four hours at 30° C., the reaction mixture was concentrated under vacuum and 4-azidobutylammonium hydrochloride was purified by column chromatography on silica gel with dichloromethane/methanol. 1.3 g (28% yield) of 4-azidodibutylammonium hydrochloride were obtained as a colourless solid.

300 MHZ-¹H-NMR (CD₃OD): δ=1.00 (t, 3H) ; 1.37-1.48 (m, 2H); 1.62-1.76 (m, 6H); 2.90-2.98 (m, 4H); 3.39 (t, 2H).

IR (KBr): v̅ (cm ⁻¹)=2932, 2870, 2797, 2453, 2091, 1593, 1462, 1261.

1. A process for the production of azidoalkylamines, characterised in that, in the first process step, a bifunctional alkane with two groups of different nucleofugicity is reacted with an azide, with the elimination of one nucleofugic group, to form a monofunctional azidoalkane and, in a second process step, the monofunctional azidoalkane is reacted with an amine with the elimination of the second nucleofugic group to form the azidoalkylamine.

2. A process according to claim 1, characterised in that the amine is a primary amine.

3. A process according to claim 1, characterised in that the azide is a metal azide with a monovalent metal from the group Li, Na, K, Rb, Cs.

4. A process according to claim 1, characterised in that the azide is a metal azide with a polyvalent metal from the group Ba, Zn, Al.

5. A process according to claim 1, characterised in that the azide is an ammonium azide, wherein up to four H atoms of the ammonium nitrogen can be substituted by organic radicals.

6. A process according to claim 1, characterised in that the azide is a metal or semimetal azide, in which the metal or the semimetal carries organic substituents.

7. A process according to claim 1, characterised in that the nucleofugic groups are selected from: chloride, bromide, iodide or sulfonates.

8. A process according to claim 1, characterised in that 1-bromo-2-chloroethane, 1-bromo-3-chloropropane, 1-bromo-3-chloro-2-methylpropane, 1-bromo-4-chlorobutane, 1-bromo-5-chloropentane, 1-bromo-6-chlorohexane, 2-bromo-1-chloropropane, 1-bromo-3-chloro-2,2-dimethoxypropane, 1-chloro-3-iodopropane, 1-chloro-4-iodobutane, 1-chloro-5-iodopentane, 1-chloro-6-iodohexane, 2-chloroethyl p-toluenesulfonate, 2-chloroethyl methanesulfonate, 3-chloropropyl p-toluenesulfonate or 2-(chloromethyl)benzyl bromide is used as the bifunctional alkane.

9. A process according to claim 1, characterised in that the first step of the process is carried out in a polar solvent.

10. A process according to claim 1, characterised in that the first step of the process is carried out in a solvent in which both the bifunctional alkane and the azide are each at least 1 wt. % soluble.

11. A process according to claim 9, characterised in that formamide, dimethylformamide, dimethylacetamide, dimethyl sulfoxide or diethylene glycol is used as the solvent.

12. A process according to claim 9, characterised in that the monofunctional azidoalkane is extracted from the polar solvent of the first step using a non-polar solvent.

13. A process according to claim 12, characterised in that water is additionally added during the extraction of the monofunctional azidoalkane after adding the non-polar solvent.

14. A process according to claim 1, characterised in that the monofunctional azidoalkane is reacted with the amine without being isolated as a pure substance.

15. A process according to claim 1, characterised in that, in the second step of the process, the amine is used in an excess of 1 to 5000 mole %.

16. A process according to claim 15, characterised in that, in the second step of the process, the amine is used in an excess of 10 to 100 mole %.

17. A process according to claim 1, characterised in that the amine contains one or more additional functional groups.

18. A process according to claim 1, characterised in that the amine is a diamine.

19. A process according to claim 18, characterised in that the diamine is used in an excess of 100 to 1000 mole %.

20. A process according to claim 1, characterised in that before, during or after the reaction with the amine, water is added or an aqueous amine is used.

21. A process according to claim 20, characterised in that the quantity of water present during the reaction is 5 to 90 wt. %, based on the total quantity of amine and water.

22. A process according to claim 20, characterised in that the quantity of water present during the reaction is 15 to 50 wt. %, based on the total quantity of amine and water.

23. A process according to claim 1, characterised in that the azidoalkylamine is an N-(azidoalkyl)diaminoalkane and is separated from the excess amine by extraction with a non-polar solvent.

24. A process according to claim 1, characterised in that the azidoalkylamine is precipitated by adding an acid.

25. A process according to claim 24, characterised in that the azidoalkylamine is fractionally precipitated by adding an acid.

26. A process according to claim 1, characterised in that the azidoalkylamine obtained is purified by chromatography as a free base, as a salt with the acid released during the reaction in the second step or as a salt from the precipitation with an acid.

27. A process according to claim 1, characterised in that the azidoalkylamine obtained is separated and/or purified by distillation.
